(19) United States
(12) Patent Application Publication
(10) Pub. No.: US 2003/0035806 A1
(43) Pub. Date: Feb. 20, 2003

(54) Novel meningitis conjugate vaccine

(76) Inventors: Anello J. D'Ambra, Mount Pocono, PA (US); Frank J. Arnold, Newfoundland, PA (US); James R. Maleckar, Nazareth, PA (US); Ronald P. McMaster, Stroudsburg, PA (US)

Correspondence Address: Aventis Pasteur, Inc., Route 611, Discovery Drive, Swiftwater, PA, 18370, US

(21) Appl. No.: 10/142,525

(22) Filed: May 9, 2002

Related U.S. Application Data
(60) Provisional application No. 60/290,200, filed on May 11, 2001.

(51) Int. Cl. A61K 39/00; A61K 39/38

(52) U.S. Cl. 424/184.1

(57) ABSTRACT

The present invention relates to immunogenic protein-polysaccharide conjugates comprising pneumococcal surface protein (PspA) obtained from Streptococcus pneumoniae conjugated to a capsular polysaccharide from N. meningitidis, and compositions comprising the same. Also provided are methods of manufacture of such immunogenic combinations as well as methods of use of such immunogenic combinations in the prevention and treatment of bacterial meningitis, particularly pneumococcal and meningococcal meningitis.

### FIELD OF THE INVENTION

The present invention relates to the fields of immunology, vaccines and the prevention of bacterial infections by immunization, more specifically to polysaccharide-protein conjugates useful as vaccines, vaccine compositions comprising two or more polysaccharide-protein conjugates and methods of vaccination using the same.

### BACKGROUND OF THE INVENTION

Neisseria meningitidis (N. meningitidis) is a leading cause of bacterial meningitis and sepsis throughout the world. Pathogenic meningococci are enveloped by a polysaccharide capsule that is attached to the outer membrane surface of the organism. N. meningitidis is classified by the immunological specificity of the capsular polysaccharide. Thirteen different serogroups of meningococci have been identified, of which five (A, B, C, W135, and Y) cause the majority of meningococcal disease. Serogroup A is responsible for most epidemic disease, which is rare in the industrialized world but occurs periodically in developing countries. Serogroups B, C, and Y cause the majority of endemic disease and localized outbreaks. Although more rare, serogroup W135 outbreaks have increased worldwide in recent years. [Samuelsson S., Eurosurveillance Weekly, 2000:17:1-5.] There are approximately 2,600 cases of bacterial meningitis per year in the United States and on average 330,000 cases in developing countries, with case fatality rates ranging between 10 and 20%.

Current meningococcal vaccines elicit an immune response against the capsular polysaccharide. The antibodies thus generated are capable of complement-mediated bacteriolysis of the serogroup specific meningococci. Meningococcal polysaccharide vaccines have been shown to be efficacious in older children and adults (Artenstein, M. S., et al. New Engl. J. Med. 282: 417-420, 1970 and Peltola, H., et al. New Engl. J. Med. 297: 686-691, 1977, but the efficacy is limited in infants and young children Reingold, A. L., et al. Lancet 2: 114-118, 1985). Subsequent doses of polysaccharide in younger populations elicited weak or no booster responses (Goldschneider, I., et al. J. Inf. Dis. 128: 769-776, 1973 and Gold, R., et al. J. Inf. Dis. 136: S31-S35, 1977). Also, the protection elicited by the meningococcal polysaccharide vaccines is not long-lasting having been estimated to be between 3 to 5 years (Brandt, B., et al. J. Inf. Dis. 131: S69-S72, 1975, and Käyhty, H., et al. J. Inf. Dis. 142: 861-868, 1980, and Ceesay, S. J., et al. J. Inf. Dis. 167: 1212-1216, 1993).

Streptococcus pneumoniae (S. pneumoniae) is another important cause of meningitis and a leading cause of fatal infections in the elderly and persons with underlying medical conditions. The most severe pneumococcal infections involve invasive meningitis and bacteremia infections, of which there are 3,000 and 50,000 cases annually, respectively. Although pneumococcal meningitis is less common than other infections caused by these bacteria, it is particularly devastating; some 10% of such patients die and greater than 50% of the remainder have life-long neurological sequelae.

Immunity to S. pneumoniae may be mediated by specific antibodies against the polysaccharide capsule of the pneumococcus. However, there are over ninety known capsular serotypes of S. pneumoniae, of which twenty-three account for about 95% of the disease. These 23 polysaccharide types have been used in a licensed pneumococcal vaccine since 1983 (Fedson, D. S., and D. M. Musher. 1994. Pneumococcal Vaccine. In Vaccines. S. A. Plotkin and J. E. A. Montimer, Eds. W. B. Saunders Co., Philidelphia, Pa., p. 517-564.). The licensed twenty-three-valent polysaccharide vaccine has a reported efficacy of approximately 60% in preventing bacteremia-caused vaccine type pneumococci in healthy adults.

However, the efficacy of the vaccine has been controversial. Antigenic competition among the twenty-three antigens may account for the poor efficacy observed. The efficacy is also affected by the fact that the twenty-three serotypes encompass all serological types associated with human infections and carriage. Also, it is not effective in children less than 2 years of age because of their inability to make adequate responses to most polysaccharides. Thus, there exists a need for a vaccine capable of conferring broad, long-lasting protection against major causes of bacterial meningitis in children.

PspA (also known as pneumococcal surface protein A or pneumococcal fimbrial protein A) is a virulence factor and protective antigen that is found in S. pneumoniae from any clade. PspA is a highly variable surface protein ranging from about 67 to 99 kDa, depending on the strain, but sufficient homologies have been identified to allow for the grouping of pneumococcal isolates into discrete sets of families and discrete sets of clades. A description of PspA from various clades and families is found in U.S. Pat. No. 5,955,089, which is incorporated herein by reference.

PspA is characterized by four domains, which include an antigenic N-terminal part, followed by a highly flexible, proline-rich region, a repeat region responsible for attachment to choline residues, and a C-terminal hydrophobic tail. The antigenic region of PspA is the N-terminal region, as determined by its ability to elicit host protective antibodies. McDaniel, L. S., et al., Microb. Pathog. 17:323-337 (1994) and Jedrzejas, M. J., et al., J. Biol. Chem. 276(35):33121-33128 (2001).

PspA may be obtained by purification from viral stocks, but large scale preparation and purification presents some difficulties and may result in impurities, additional expense and variability in the PspA obtained. It is more desirable, from a vaccine manufacturing perspective, to produce PspA by recombinant means, such as an expression vector pET-9a used to transform Escherichia coli (E. coli) BL21. Jedrzejas, M. J., et al., J. Biol. Chem. 276(35):33121-33128 (2001). Recombinant technology may also be used to generate truncated or modified forms of truncated PspA.

The term PspA/Rx1 refers to a truncated PspA protein comprising the N-terminal region (approximately amino acids 1-314) of the naturally occurring PspA polypeptide of strain Rx1 of S. pneumoniae. Jedrzejas, M. J., et al., J. Biol. Chem. 276(35):33121-33128 (2001).

### SUMMARY OF THE INVENTION

The present invention provides immunogenic protein-polysaccharide conjugates comprising pneumococcal surface protein (PspA) obtained from Streptococcus pneumoniae conjugated to a capsular polysaccharide from N. meningitidis. The immunogenic compositions of the present invention include full-length PspA as well as immunogenic PspA fragments, and modified forms of PspA. N-terminal fragments of PspA are preferred in the protein-polysaccharide conjugates of the instant invention.

The present invention provides vaccines comprising a protein-polysaccharide conjugate comprising a PspA protein obtained from S. pneumoniae conjugated to a capsular polysaccharide from N. meningitidis.

The present invention also provides vaccines comprising one or more, preferably from two to seven, protein-polysaccharide conjugate each comprising a PspA protein selected from a clade of S. pneumoniae conjugated to a capsular polysaccharide selected from one or more serogroups of N. meningitidis. Another embodiment, the invention provides vaccines comprising one or more, preferably from two to seven, protein-polysaccharide conjugate wherein the PspA is a fragment comprising the N-terminal portion of PspA, and alternatively, comprising the N-terminal and all or part of the proline-rich region of PspA. In a preferred embodiment, the PspA fragment comprises an N-terminal portion of the strain R36A, or variants thereof, including Rx1314. A more preferred embodiment comprises a PspA variant of a fragment of Rx1314, Rx1314MI, comprising amino acids 1 to 314 wherein the methionine at amino acid position 96 is modified to isoleucine.

The present invention also provides methods of manufacture of meningitis vaccines based on immunogenic compositions comprising a PspA protein from S. pneumoniae conjugated to a polysaccharide from N. meningitidis. Another embodiment of the invention provides methods of manufacture of vaccines comprising one or more, preferably from two to seven, protein-polysaccharide conjugate wherein the PspA is a fragment comprising the N-terminal portion of PspA, and alternatively, comprising the N-terminal and all or part of the proline-rich region of PspA. In a preferred embodiment, the PspA fragment comprises an N-terminal portion of the strain R36A, or variants thereof, including Rx1314. A more preferred embodiment comprises a PspA variant of a fragment of Rx1314, Rx1314MI, comprising amino acids 1 to 314 wherein the methionine at amino acid position 96 is modified to isoleucine.

The invention also provides a method of inducing an immunological response in a host mammal comprising administering to the host an immunogenic, immunological or vaccine composition comprising a PspA-meningococcal polysaccharide conjugate and a pharmaceutically acceptable carrier or diluent.

Throughout this specification, reference is made to various documents so as to describe more fully the state of the art to which this invention pertains. These documents, as well as documents cited by these documents, are each hereby incorporated by reference.

### DETAILED DESCRIPTION OF THE INVENTION

The present invention provides a protein-polysaccharide conjugate comprising a PspA protein conjugated to a meningococcal polysaccharide, methods of manufacture of vaccines comprising the same and immunogenic compositions capable of conferring broad, long-lived protection against major pathogenic strains of N. meningitidis. PspA protein from S. pneumoniae can be conjugated to meningococcal polysaccharide to serve as a carrier protein for converting the polysaccharide to T-dependent antigens while at the same time engendering an antibody response against the PspA carrier protein that may help protect against S. pneumoniae since immunologically important epitopes within the PspA carrier proteins are not affected by the conjugation process.

The invention includes a protein-polysaccharide conjugate comprising a PspA protein conjugated to a meningococcal polysaccharide. The PspA protein is selected from a lade of S. pneumoniae. In one embodiment, the protein-polysaccharide conjugate comprises a PspA fragment lacking the C-terminal portion of PspA. In another embodiment, the protein-polysaccharide conjugate comprises a PspA fragment lacking the C-terminal portion and repeat region of Psp-A. In another embodiment, the protein-polysaccharide conjugate comprises a PspA fragment lacking the C-terminal portion, the repeat region and all or part of the proline-rich region of PspA. Alternatively, the protein-polysaccharide conjugate comprises the N-terminal portion of PspA, or comprises the N-terminal and all or part of the proline-rich region of PspA.

In one aspect of the invention, the PspA fragment comprises an N-terminal portion of a PspA protein selected from clades 1, 2, 3, 4, 5, or 6 of S. pneumoniae. In another aspect of the invention, the PspA fragment comprises an N-terminal portion of a PspA strain selected from clades 1, 2 or 3 of S. pneumoniae. In yet another aspect of the invention, the PspA fragment comprises an N-terminal portion of a PspA strain selected from clades 4, 5 or 6 of S. pneumoniae. In a more preferred embodiment of the invention, the PspA fragment comprises an N-terminal portion of a PspA strain selected from clade 2 of S. pneumoniae. In an even more preferred embodiment of the invention, the PspA fragment comprises an N-terminal portion of a PspA from the strain R36A, or variants thereof, including Rx1314. A more preferred embodiment comprises a PspA variant of a fragment of Rx1314, Rx1314MI, comprising amino acids 1 to 314 wherein the methionine at amino acid position 96 is modified to isoleucine. In another embodiment, the meningococcal polysaccharide is selected from a capsular polysaccharide. In a preferred emobdiment, the polysaccharide is selected from serogroups A, C, Y, W-135 of N. meningitidis. In another embodiment of the invention, the meningococcal polysaccharide is selected from clades 1, 2, 3, and 5 of S. pneumoniae. More preferably, the PspA protein(s) conjugated to the meningococcal polysaccharides is selected from clades 1, 2, 3, 5 and 6 of S. pneumoniae.

In a preferred emobdiment, the polysaccharide is selected from serogroups A, C, Y, W-135 of N. meningitidis and the meningococcal polysaccharide is selected from clades 1, 2, 3, and 5 of S. pneumoniae. More preferably, the PspA protein(s) conjugated to the meningococcal polysaccharides is selected from clades 1, 2, 3, 5 and 6 of S. pneumoniae. In another embodiment of the invention meningococcal polysaccharides conjugated to the PspA protein(s) is selected from serogroups A, C, Y, and W-135 of N. meningitidis., the PspA is selected from clades 1, 2, 3, and 5 of S. pneumoniae. More preferably, the PspA protein(s) conjugated to the meningococcal polysaccharides is selected from clades 1, 2, 3, 5 and 6 of S. pneumoniae.

In a preferred embodiment of the present invention the immunogenic composition comprises from 3 to 5 capsular polysaccharides from different serogroups of N. meningitidis conjugated to 2 to 4 PspA proteins from different clades of S. pneumoniae. Preferably, the Preferably, the PspA protein(s) conjugated to the meningococcal polysaccharides is selected from clades 1, 2, 3, and 5 of S. pneumoniae. More preferably, the PspA protein(s) conjugated to the meningococcal polysaccharides is selected from clades 1, 2, 3, 5 and 6.

In the examples of the present embodiment of the invention, contained herein, describe processes, which include the conjugation of the four N. meningitidis serogroups, A, C, Y, and W-135, of to lade 2 of PspA, namely, Rx1-MI described above. No one meningococcal polysaccharide from a particular serogroup need be conjugated to a PspA protein from a particular clade. Thus, in immunogenic compositions of the present invention, meningococcal polysaccharides from serogroup C of N. meningitidis could be conjugated to PspA proteins from clades 1-7. The present invention also provides for immunogenic compositions in which meningococcal polysaccharides from a single serogroup could be conjugated to PspA proteins from more than one S. pneumoniae lade. For example, in immunogenic compositions of the present invention meningococcal polysaccharides from serogroup C of N. meningitidis could be conjugated to PspA proteins from both clades 1 and 2.

PspA (also known as pneumococcal surface protein A or pneumococcal fimbrial protein A) is a virulence factor and protective antigen that is found in S. pneumoniae from any lade. In the immunogenic compositions of the present invention PspA proteins are used as carrier proteins for the meningococcal polysaccharides. PspA is a highly variable surface protein, but sufficient homologies have been identified to allow for the grouping of pneumococcal isolates into discrete sets of families and discrete sets of clades. A description of PspA from various clades and families is found in U.S. Pat. No. 5,955,089, which is incorporated herein by reference. The immunogenic compositions of the present invention include full-length PspA as well as immunogenic PspA fragments. The term PspA/Rx1 refers to a truncated PspA protein comprising the N-terminal region (approximately amino acids 1-314) of the naturally occurring PspA polypeptide of strain Rx1 of S. pneumoniae.

PspA/Rx1-MI refers to a truncated PspA protein which is the N-terminal region (approximately amino acids 1-314) of the naturally occurring PspA polypeptide of strain Rx1 of S. pneumoniae in which the first internal methionine from the N-terminus (amino acid 96 in the native sequence) has been changed to isoleucine. M J Jedzrejas, M. J., et al., J. Biol. Chem., 276 (2001):33121-33128, incorporated herein by reference. A number of U.S. patents and patent applications, including U.S. Pat. No. 5,955,089; U.S. Ser. No. 08/529,055, filed Sep. 15, 1995; U.S. Ser. No. 08/470,626, filed Jun. 6, 1995; U.S. Pat. No. 5,856,170; U.S. Pat. No. 5,753,463; U.S. Ser. No. 08/468,718, filed Jun. 6, 1995 [U.S. Pat. No. 5,871,943]; U.S. Ser. No. 08/247,491, filed May 23, 1994 [U.S. Pat. No. 5,965,400]; U.S. Pat. No. 5,728,387; U.S. Ser. No. 08/214,164, filed Mar. 17, 1994 [U.S. Pat. No. 5,728,387]; U.S. Ser. No. 08/246,636, filed May 20, 1994 [U.S. Pat. No. 5,965,141]; U.S. Ser. No. 08/319,795, filed Oct. 7, 1994 [U.S. Pat. No. 5,980,909]; and U.S. Pat. No. 5,476,929, relate to vaccines comprising PspA and fragments thereof, methods for expressing DNA encoding PspA and fragments thereof, DNA encoding PspA and fragments thereof, the amino acid sequences of PspA and fragments thereof, compositions containing PspA and fragments thereof and methods of using such compositions. The teachings of these applications are relevant to the present invention and these applications, together with any and all of the references cited therein, are incorporated herein by reference.

Strains of S. pneumoniae corresponding to clade 1 include, by way of example and not limitation, AC94, BG6692, BG 8743, BG8838, DBL1 DBL6A, BG9739 (ATCC 55837) and L81905. Strains corresponding to lade 2 include Rx1 (ATCC 55834), EF10197, WU2, 0922134, DBL5, BG9163, EF6796, RCT123, RCT129, RCT135 and LXS200. Strains corresponding to lade 3 include EF3296 (ATCC 55835), AC122 and BG8090. Strains corresponding to clade 4 include EF5668 (ATCC 55836), BG7817, BG7561 and BG11703. Strains corresponding to clade 5 include ATCC 6303, and strains corresponding to clade 6 include BG6380 (ATCC 55838). Again, the information provided in U.S. Pat. No. 5,955,089 can be used to determine the lade number of any strain of S. pneumoniae, such that PspA from any such strain can be identified with a specific clade. This, in turn, allows one to select PspA carrier proteins corresponding to clades of interest so as to formulate conjugates capable of eliciting protection against most strains of S. pneumoniae. In addition, the PspA used in the present invention may be fragments of the PspA derived from any of the above strains corresponding to clades 1 to 6 above, wherein the PspA is a fragment comprising the N-terminal portion of PspA, and alternatively, comprising the N-terminal and all or part of the proline-rich region of PspA. In a preferred embodiment, the PspA fragment comprises an N-terminal portion of the strain R36A, or variants thereof, including Rx1314. A more preferred embodiment comprises a PspA variant of a fragment of Rx1314, Rx1314MI, comprising amino acids 1 to 314 wherein the methionine at amino acid position 96 is modified to isoleucine. The recombinant PspA construct Rx1314MI is hereafter also referred to as Rx1-MI.

The immunogenic compositions of the present invention are made by conjugating PspA(s) to meningococcal polysaccharide. The PspA(s) to be conjugated to meningococcal polysaccharide may be purified from wild-type S. pneumoniae or produced recombinantly. The preferred PspA(s) immunogenic compositions of the present invention are those that have been produced as recombinant because they can be produced at high yield and purity. Molecular cloning and expression of PspA from any given lade can be accomplished by one of skill in the art, using standard molecular biological and biochemical techniques. The sequence of PspA, provided, for example, in U.S. Pat. No. 5,476,929 can be used to prepare oligonucleotide probes. Such probes can be used, together with the teachings of U.S. Pat. No. 5,955,089 to clone the gene for PspA from any desired strain of S. pneumoniae, using standard techniques known in the art, for example, as described by Maniatis et. al., Molecular Cloning—A Laboratory Manual, 1982, Cold Spring Harbor Laboratory, or Sambrook et. al., Molecular Cloning, 2^{nd} ed., 1989, Cold Spring Harbor Laboratory.

Polysaccharides used to prepare the conjugates of the present invention can be readily prepared from any bacterium of interest, preferably from serogroups A, C, W-135 and/or Y of N. meningitidis. Polysaccharides may be obtained from commercial sources or prepared by any number of standard techniques, typically involving chemical extraction or enzymatic hydrolysis of whole cells. The preferred method for making the polysaccharides used in the present invention is described in U.S. Pat. No. 4,123,520, incorporated by reference. Protein-polysaccharide conjugates of the present invention can be prepared by any number of direct or indirect (i.e., using a linker molecule) coupling chemistries. (See, e.g., U.S. Pat. Nos. 4,057,685; 4,356,170; 4,459,286; 4,496,538; 4,619,828; 5,306,492; 5,693,326; and 6,309,646.) Conjugation methods applicable to the present invention include, by way of example and not limitation, reductive amination, diazo coupling, thioether bond, disulfide bond, amidation and thiocarbamoyl chemistries. (See, e.g., W. E. Dick and M. Beurret, 1989, Glycoconjugates of bacterial carbohydrate antigens, pg. 48-114. In: Conjugate Vaccines Contributions to Microbiology and Immunology. J. M. Cruse and R. E. Lewis, Jr. (eds.), S. Karger, Basel).

The immunogenic compositions of the present invention are not limited to conjugates of PspA(s) and meningococcal polysaccharide(s). The immunogenic compositions of the present invention also include compositions in which unconjugated PspA protein could be added to the immunogenic composition, or that the composition in its final form could contain other antigens such as Haemophilus influenzae type b conjugate vaccine. Conjugates comprising bacterial polysaccharides such as PRP from Haemophilus influenzae type b (Hib) conjugated to PspA are also included within the scope of the present invention. Those skilled in the art will recognize that any number of different conjugation chemistries could be used to generate the polysaccharide-PspA conjugates of the present invention.

The invention also provides a method of inducing an immunological response in a host mammal comprised of administering to the host an immunogenic, immunological or vaccine composition comprising PspA-meningococcal polysaccharide conjugates and a pharmaceutically acceptable adjuvant or diluent. The determination of the amount of each PspA carrier and meningococcal polysaccharide within each PspA-meningococcal polysaccharide conjugate, as well as the amount of each PspA-meningococcal polysaccharide conjugate and optional additional adjuvant in the inventive compositions and the preparation of those compositions can be in accordance with standard techniques well known to those skilled in the pharmaceutical or veterinary arts. In particular, the amount of protein-polysaccharide conjugate and adjuvant in the inventive compositions and the dosages administered are determined by techniques well known to those skilled in the medical or veterinary arts taking into consideration such factors as the particular antigen, the adjuvant (if present), the age, sex, weight, species and condition of the particular animal or patient, and the route of administration. Thus, the skilled artisan can readily determine the amount of conjugate and optional adjuvant in compositions and to be administered in methods of the invention. The antibody response in an individual can be monitored by assaying for antibody titer, bactericidal activity or opsonophagocytic activity and boosted if necessary to enhance the response. Typically, a single dose for an infant is about 1-30 μg of conjugate (for each serogroup) vaccine per dose given intramuscularly.

The vaccines of the present invention may include an adjuvant or adjuvants and stabilizers to enhance the immunological response of the recipient and maintain integrity of the product. Typically, adjuvants and stabilizers are commonly used as 0.0005 to 50 wt % solution in phosphate-buffered saline (PBS). The conjugate is present on the order of micrograms to milligrams, such as about 0.000001 to about 5 wt %, preferably about 0.000001 to about 1 wt %, most preferably about 0.0001 to about 0.05 wt % (see, e.g., Examples below or in applications cited herein). Such adjuvants may be aluminum based such as aluminum phosphate or aluminum hydroxide. Several alternative adjuvants are known in the art, for example, as described in M. F. Powell and M. J. Newman, eds., Vaccine Design, the Subunit and Adjuvant Approach, 1995, Plenum Press, NY.

Examples of compositions of the invention include liquid preparations for orifice, e.g., oral, nasal, anal, vaginal, peroral, intragastric, mucosal (e.g., perlingual, alveolar, gingival, olfactory or respiratory mucosa) etc., administration such as suspensions, syrups or elixirs; and, preparations for parenteral, subcutaneous, intradermal, intramuscular or intravenous administration (e.g., injectable administration), such as sterile suspensions or emulsions. Such compositions may be in admixture with a suitable carrier, diluent, or excipient such as sterile water, physiological saline, glucose or the like. The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as Remingon's Pharmaceutical Science, 17th edition, 1985, incorporated herein by reference, may be consulted to prepare suitable preparations, without undue experimentation.

Liquid preparations are normally easier to prepare than gels, other viscous compositions, and solid compositions. Additionally, liquid compositions are somewhat more convenient to administer, especially by injection or orally, to animals, children, particularly small children, and others who may have difficulty swallowing a pill, tablet, capsule or the like, or in multi-dose situations. Viscous compositions, on the other hand, can be formulated within the appropriate viscosity range to provide longer contact periods with mucosa, such as the lining of the stomach or nasal mucosa.

The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

A pharmaceutically acceptable preservative can be employed to increase the shelf-life of the compositions. Benzyl alcohol may be suitable, although a variety of preservatives including, for example, parabens, chlorobutanol, or benzalkonium chloride may also be employed. A suitable concentration of the preservative will be from 0.02% to 2% based on the total weight although there may be appreciable variation depending upon the agent selected.

Those skilled in the art will recognize that the components of the compositions must be selected to be chemically inert with respect to the antigens and optional additional adjuvant.

The present invention is additionally described by way of the following illustrative, non-limiting examples.

### EXAMPLES

### Example 1

### Preparation of PspA Carrier Proteins

Master and working seed preparation. E. coli BL21 (DE3) competent cells were transformed using purified plasmid pET-9a (Novagen). The competent cells and plasmid were mixed with LB medium and incubated 35 to 38° C. for one hour. Cultures were streaked onto LB+kanamycin agar plates and incubated for 16 to 24 hours at 35-38° C. Colonies were harvested and resuspended in LB medium and incubated for 6 hours at 35-38° C. Sterile glycerol, 50%, was added and cultures were harvested, sealed into vials, and stored at ≦60° C. Working seeds were prepared from the master seed by inoculating LB medium containing kanamycin with the master seed culture for 6 hours at 35-38° C. Sterile glycerol, 50%, was added and cultures were harvested, sealed into vials, and stored at ≦−60° C.

Preparation of crude pneumococcal surface protein A. Working seed was thawed and used to inoculate a shaker flask containing 1 L of chemically defined medium, supplemented with kanamycin and magnesium sulfate. The culture was incubated for 15 to 20 hours at 37° C. The contents of the shaker flask was transferred to a fermenter containing chemically defined medium supplemented with kanamycin and magnesium sulfate. Fed-batch fermentation was conducted at constant temperature of 37° C. Dissolved oxygen was controlled at 30% air saturation, and pH was maintained at 6.9. A solution of antifoam was added when needed to suppress any foaming that occurred during the fermentation. A portion of the seed culture fermenter was used to inoculate a production fermenter containing chemically defined medium that was supplemented with kanamycin and magnesium sulfate. Fermentation was allowed to proceed maintaining constant temperature and pH. After approximately 4 to 5 hours nutrient feed was added at a constant feed rate. When the optical density reached 8 to 10, the inducer, isopropyl □-D-thiogalactoside (IPTG), was added, and fermentation was continued for approximately 8 hours. The culture was harvested from the fermenter by microfiltration. The filtrate was passed through a mechanical homogenizer twice in order to prepare lysate. The lysate was clarified using a disc-staked centrifuge. The clarified lysate was concentrated and diafiltered (10,000 MWCO membrane) into 15 mM Tris buffer, pH 8.0, containing 2 mM EDTA. The material was filtered through a 0.8-μm membrane and sterilized by filtration through a 0.2-μm membrane.

Purification of pneumococcal surface protein A. Following sterile filtration of the crude fermented broth preparation, the material is loaded onto an anion exchange column previously equilibrated in 15 mM citrate, pH 6.0 buffer. Once the material is loaded onto the column, the column is further washed with the equilibration buffer to remove unbound contaminating materials. After washing the column is eluted with 0.1 M NaCl in 15 mM citrate pH 6.0. The resulting eluate is dialyzed into 15 mM Tris pH 7.0 buffer and ammonium sulfate is added to a final concentration of 2.0 M. The material then undergoes a clarification step utilizing depth filtration (nominal porosity 0.8 μm). The clarified material is then applied to a hydrophobic interaction column (e.g. packed with Fractogel Propyl (S), Merck KGaA) that was previously equilibrated in 2.0 M ammonium sulfate with 15 mM Tris pH 7.0. The partially purified PspA is applied to the column and eluted with 1.2 M ammonium sulfate in 15 mM Tris, pH 7.0. The resulting purified PspA is dialyzed into a buffer suitable for storage such as PBS or Tris-buffered saline (TBS), pH 7.2.

### Example 2

### Preparation of Monovalent Conjugates of Neisseria meningitidis Serogroup a Polysaccharide to Pneumococcal Surface Protein a Carrier Proteins

The chemical modification steps for group A conjugation are based on the process described in U.S. Pat. No. 5,965,714, incorporated herein by reference. Native group A polysaccharide isolated by the process described in U.S. Pat. No. 4,123,520 was dissolved in sodium at an approximate concentration of 10 mg/ml. To this solution hydrogen peroxide (to 0.5-20% final concentration) and mild acid (pH 5-6) was added. Oxidative depolymerization was allowed to proceed and was monitored by high-performance size-exclusion chromatography (HPSEC) to give a sized polysaccharide in the range of 10,000 daltons to 300,000 daltons, more preferably 50,000 daltons to 200,000 daltons. Once the saccharide falls within the desired range, the activated saccharide (diluted to 4-6 mg/ml) was derivatized with a dihydrazide cross-linking agent, preferably adipic dihydrazide (0.5-2 mg/ml), in the presence of EDAC (0.5-2 mg/ml) and sodium cyanoborohydride (1-4 mg/ml). Small polysaccharide fragments, unreacted reagents, and reagent by-products were removed via ultrafiltration (10 k MWCO membrane) into saline. The sized, activated, and now-derivatized polysaccharide concentrate (1-40 mg/ml) was reacted with the PspA protein (0.1-40 mg/ml) via amidation in the presence of N-(3-dimethylaminopropyl)-N′-ethylcarbodiimide hydrochloride (EDAC) (0.5-2 mg/mg protein) at pH 5 to 6. The reaction proceeds to completion, as determined by HPSEC and the pH of reaction mixture was then adjusted to 6.5-7 to stop any side reactions. The resulting material constitutes the crude conjugate which is further purified to remove process impurities and unreacted protein and polysaccharide. The crude conjugate was desalted by diafiltration (10 k MWCO membrane) into saline. Ammonium sulfate was added to 2 M final concentration to the retentate for preparation to purify material via hydrophobic interaction chromatography (HIC) (e.g on Fractogel Phenyl (S), Merck KGaA). After loading of the crude conjugate onto the column, it was eluted with 2 M ammonium sulfate to remove unconjugated polysaccharide. The column was washed with ammonium sulfate at an intermediate concentration (e.g. 0.4 to 1.2 M) to ensure removal of unconjugated polysaccharide. The purified conjugate was recovered by subsequent elution of the column with water. The resulting material was subjected to additional diafiltration (100 k MWCO membrane) into saline to remove unreacted protein and ammonium sulfate. This final purified conjugate was then sterile-filtered and used for vaccine formulation.

### Example 3

### Preparation of Monovalent Conjugates of Neisseria meningitidis Serogroup C Polysaccharide to Pneumococcal Surface Protein a Carrier Proteins

Native group C polysaccharide isolated by the process described in U.S. Pat. No. 4,123,520 was dissolved in water at an approximate concentration of 10 mg/ml. To this solution was added a solution of sodium periodate to give a final concentration in the reaction of 5-50 mol %, preferably 5-20 mol %, relative to the content of polysaccharide's repeat unit (mol. wt. 345, based on partial O-acetylation). Oxidative depolymerization was allowed to proceed in the dark at 20 to 30° C. and was monitored by HPSEC. The product was sized polysaccharide in the range of 10,000 daltons to 300,000 daltons, more preferably 50,000 daltons to 200,000 daltons. The reagent and its by-products, as well as small polysaccharide fragments, were removed via diafiltration (10 k MWCO membrane) into saline. The sized, activated polysaccharide concentrate (1-40 mg/ml) is reacted with the PspA protein (0.1-40 mg/ml) via reductive amination in the presence of sodium cyanoborohydride (1 mg/mg protein). The progress of the reaction was monitored by HPSEC. Upon completion, the reaction is quenched with excess sodium borohydride. The resulting material constitutes the crude conjugate, which is further purified to remove process impurities and unreacted protein and polysaccharide. The crude conjugate is desalted by diafiltration (10 k MWCO membrane) into saline. Ammonium sulfate is added to 1 M final concentration to the retentate for preparation to purify material via HIC (e.g., on Fractogel Phenyl (S)). After loading of the crude conjugate onto the column, it was eluted with 1 M ammonium sulfate to remove unconjugated polysaccharide. The purified conjugate was recovered by subsequent elution of the column with water. The resulting material was subjected to additional diafiltration (100 k MWCO membrane) into saline to remove unreacted protein and ammonium sulfate. This final purified conjugate was then sterile-filtered and used for vaccine formulation.

### Example 4

### Preparation of Monovalent Conjugates of Neisseria meningitidis Serogroup W135 Polysaccharide to Pneumococcal Surface Protein a Carrier Proteins

Native group W135 polysaccharide isolated by the process described in U.S. Pat. No. 4,123,520 was dissolved at an approximate concentration of 10 mg/ml in mild acid, preferably sodium acetate buffer, pH 4.8-5.2, and hydrolytic depolymerization was allowed to proceed at 50-80° C. The progress of the reaction was monitored by HPSEC The resulting sized polysaccharide, in the range of 10,000 daltons to 300,000 daltons, more preferably 50,000 daltons to 200,000 daltons, was then activated at 20-30° C. with sodium periodate (1-50 mole %, relative to the content of polysaccharide's repeat unit (mol. wt. 503, based on partial O-acetylation)). The reagent and its by-products, as well as small polysaccharide fragments, were removed via diafiltration (10 k MWCO membrane) into saline. The sized, activated polysaccharide concentrate (1-40 mg/ml) was reacted with the PspA protein (0.1-40 mg/ml) via reductive amination in the presence of sodium cyanoborohydride (1 mg/mg protein). The progress of the reaction was monitored by HPSEC. Upon completion, the reaction is quenched with excess sodium borohydride. The resulting material constitutes the crude conjugate, which is further purified to remove process impurities and unreacted protein and polysaccharide. The crude conjugate was desalted by diafiltration (10 k MWCO membrane) into saline. Ammonium sulfate was added to 1 M final concentration to the retentate for preparation to purify material via HIC (e.g., on Fractogel Phenyl (S)). After loading of the crude conjugate onto the column, it was eluted with 1 M ammonium sulfate to remove unconjugated polysaccharide. The purified conjugate was recovered by subsequent elution of the column with water. The resulting material was subjected to additional diafiltration (100 k MWCO membrane) into saline to remove unreacted protein and ammonium sulfate. This final purified conjugate was then sterile-filtered and used for vaccine formulation.

### Example 5

### Preparation of Monovalent Conjugates of Neisseria meningitidis Serogroup Y Polysaccharide to Pneumococcal Surface Protein a Carrier Proteins

Native group Y polysaccharide isolated by the process described in U.S. Pat. No. 4,123,520 was dissolved at an approximate concentration of 10 mg/ml in mild acid, preferably sodium acetate buffer, pH 4.8-5.2, and hydrolytic depolymerization was allowed to proceed at 50-80° C. The progress of the reaction was monitored by HPSEC The resulting sized polysaccharide, in the range of 10,000 daltons to 300,000 daltons, more preferably 50,000 daltons to 200,000 daltons, was then activated at 20-30° C. with sodium periodate (1-50 mole %, relative to the content of polysaccharide's repeat unit (mol. wt. 504, based on partial O-acetylation)). The reagent and its by-products, as well as small polysaccharide fragments, were removed via diafiltration (10 k MWCO membrane) into saline. The sized, activated polysaccharide concentrate (1-40 mg/ml) was reacted with the PspA protein (0.1-40 mg/ml) via reductive amination in the presence of sodium cyanoborohydride (1 mg/mg protein). The progress of the reaction was monitored by HPSEC. Upon completion, the reaction was quenched with excess sodium borohydride. The resulting material constitutes the crude conjugate, which is further purified to remove process impurities and unreacted protein and polysaccharide. The crude conjugate is desalted by diafiltration (10 k MWCO membrane) into saline. Ammonium sulfate was added to 1 M final concentration to the retentate for preparation to purify material via HIC (e.g., on Fractogel Phenyl (S)). After loading of the crude conjugate onto the column, it was eluted with 1 M ammonium sulfate to remove unconjugated polysaccharide. The purified conjugate was recovered by subsequent elution of the column with water. The resulting material was subjected to additional diafiltration (100 k MWCO membrane) into saline to remove unreacted protein and ammonium sulfate. This final purified conjugate was then sterile-filtered and used for vaccine formulation.

### Example 6

### Physicochemical Characteristics and Antigenicity of Meningococcal Polysaccharide-PspA Conjugates

The physicochemical characteristics of representative meningococcal conjugates made with PspA Rx1-MI are given in Table 1. Polysaccharide content was determined by calorimetric assays for sialic acid (groups C, W135, and Y; Svennerholm, L., Biochem Biophys. Acta 24:604-611, 1957) or for phosphorus (group A; Bartlett, G. R., J. Biol. Chem. 234:466-468, 1958). These colorimetric measurements allow the calculation of the mass ratio of polysaccharide to protein in the conjugate as given in Table 1. Content of residual unbound polysaccharide (“free polysaccharide”) was determined from calorimetric analysis of fractions obtained by solid-phase extraction using disposable cartridges pre-packed with a reversed-phase chromatographic medium (SPICE C-2, Analtech) to separate unbound polysaccharide from conjugate. Protein was determined using the commercially available Pierce BCA® assay kit. Content of O-acetyl (O-Ac) groups was determined by the method of Hestrin (Hestrin, S., J Biol. Chem. 180:879-881, 1949). Molecular weight (“Mol. Wt.”) of the depolymerized polysaccharide was determined by HPSEC on an instrument equipped with a differential refractometry (RI) detector and a Waters Ultrahydrogel® 500 column, eluted with phosphate-buffered saline (PBS); the column was calibrated with dextran molecular-weight standards. Molecular weight of the conjugates (given as the weight-averaged molar mass, M_{w}) was determined by HPSEC with detection by multiangle light-scattering photometry and differential refractometry (HPSEC-MALS/RI) on an instrument system equipped with a Wyatt Technology DAWN® MALS detector and a Phenomenex BioSep-SEC-S 4000 column, eluted with PBS. The specific refractive-index increment (dn/dc) was assigned the value 0.160 for conjugates; polydispersity, as given in Table 1, is the ratio of M_{w} to the number-averaged molar mass (Mₙ), and is an indicator of the size heterogeneity of the conjugate. Content of residual unbound polysaccharide (“free PS”) was determined from calorimetric analysis of fractions obtained by separation of conjugate from unbound polysaccharide on reverse-phase solid-phase extraction cartridges (Analtech C-2). Residual unbound protein (“free protein”) was determined by integration of the ultraviolet signal, monitored at 280 nm, obtained by HPSEC of the conjugate product using the same chromatography system as that used for molecular-weight determination of the conjugates.

Antigenicity of the immunogens was analyzed by rate nephelometry, which measures the rate of formation of immune complexes in solution by detection of changes in the intensity of scattered light. Antigenicity of conjugate components is expressed as percentages relative to rates obtained for their respective reference standards (native meningococcal polysaccharides or PspA) using antisera raised against whole meningococci of each serogroup or against a particular native or mutant strain of PspA, e.g. Rx1-MI.

The characterization data shown in Table 1 indicate that conjugates can be prepared with PspA as the carrier protein by processes which preserve the structural integrity of the polysaccharide antigens and effectively remove unreacted components.

TABLE 1
Physiochemical Characteristics and Antigenicity of Representative Meningococcal
Polysacchraide-PspA/Rx1-MI Conjugates

| | Mol. | | PS/protein | | | | % Anti- | |
|---|---|---|---|---|---|---|---|---|
| | Wt. (kg/mol) | | (by | | Free | O-Ac | genicity⁶ | |
| Serogroup | PS^{1,2} | Conj.³ | mass) | Free PS | protein | (μmol/mg PS) | PS | PspA |
| A | 180 | 494 | 1.3 | 13% | ND⁵ | 2.8 | 80 | ND |
| | | (1.32) | | | | | | |
| C | 170 | 360 | 2.1 | <2%⁴ | ND | 2.1 | >100 | 62 |
| | | (1.42) | | | | | | |
| W-135 | 190 | 349 | 2.0 | <2%⁴ | ND | 1.2 | >100 | 38 |
| | | (1.29) | | | | | | |
| Y | 170 | 366 | 1.7 | <2%⁴ | ND | 1.3 | 75 | 39 |
| | | (1.26) | | | | | | |

¹PS, polysaccharide.
²Mol. Wt. relative to dextran.
³Mol. Wt. by HPSEC-MALS, polydispersity in parentheses.
⁴Less than lower limit of quantitation.
⁵ND, Not detected.
⁶Relative to native polysaccharide (PS) or free PspA/Rx1-MI (PspA).

### Example 7

### Immunogenicity of Meningococcal Polysaccharide-Rx1-MI Conjugates in Mice

The immune response to the meningococcal polysaccharide-PspA conjugates described in the examples above was investigated in mice. Mice (BALB/c, female, 6-8 week old; 10 mice per treatment group) were injected subcutaneously with vaccine formulations (containing 0.25 μg polysaccharide per serogroup per dose as applicable), as designated in Table 2, on days 0 and 21 and bled on days 0 (untreated group only), 20 and 42. Sera were analyzed for anti-polysaccharide or anti-Rx1-MI IgG by ELISA. Selected data from the study are given in Table 2. The antibody responses to unconjugated polysaccharides in BALB/c mice are typically equivalent to the responses obtained in the non-immunized control group; therefore, formulations containing unconjugated polysaccharides were not used as test articles in this study. The results clearly show that the anti-polysaccharide response in mice was improved because of conjugation to PspA. Furthermore, the anti-PspA response in mice due to conjugation was demonstrated in this study not to be significantly different from the response to PspA alone.

TABLE 2
Antibody Response in BALB/C Mice to Meningococcal
Polysaccharide-PspA Conjugates

| | Geometric mean IgG response¹ | |
|---|---|---|
| | Men PS | PspA/Rx1-MI |
| Immunogen² | (ELISA units/mL) | (μg IgG/mL) |
| MenA PS-PspA/Rx1-MI | 17520 | 26.2³ |
| MenA PS-PspA/Rx1-MI + adj.⁴ | 29240 | 68.4³ |
| MenC PS-PspA/Rx1-MI | 6200 | — |
| MenC PS-PspA/Rx1-MI + adj. | 12020 | — |
| MenW-135 PS-PspA/Rx1-MI | 6740 | — |
| MenW-135 PS-PspA/Rx1-MI + adj. | 10090 | — |
| MenY PS-PspA/Rx1-MI | 12620 | — |
| MenY PS-PspA/Rx1-MI + adj. | 25220 | — |
| PspA/Rx1-MI | NA⁵ | 1.4 |
| PspA/Rx1-MI + adj. | NA | 24.0 |
| Untreated controls | <148^{6.7} | <0.4⁶ |

¹From day-42 bleeds.
²Conjugates in tetravalent formulations. PspA/Rx1-MI in single-antigen formulations.
³Anti-Rx1-MI response for tetravalent conjugate formulations.
⁴adj., adjuvant: aluminum oxyphosphate.
⁵Not applicable.
⁶No response.
⁷Response for Group C; also, no response for all others.

What is claimed:

1. A protein-polysaccharide conjugate comprising the N-terminal fragment of a pneumococcal surface protein (PspA) obtained from Streptococcus pneumoniae conjugated to a capsular polysaccharide from N. meningitidis.

2. The protein-polysaccharide conjugate of claim 1, wherein the N-terminal fragment further comprises all or part of the proline-rich region of PspA of S. pneumoniae.

3. The protein-polysaccharide conjugate of claim 1, wherein the N-terminal fragment is selected from a PspA of clades 1, 2, 3, 4, 5, or 6 of S. pneumoniae.

4. The protein-polysaccharide conjugate of claim 3, wherein the N-terminal fragment is selected from a PspA of clades 1, 2 or 3 of S. pneumoniae.

5. The protein-polysaccharide conjugate of claim 4, wherein the N-terminal fragment is selected from a PspA of clade 2 of S. pneumoniae.

6. The protein-polysaccharide conjugate of claim 5, wherein the N-terminal fragment is selected from a strain R36A, or variants thereof, including strains Rx1314, Rx1 (ATCC 55834), EF10197, WU2, 0922134, DBL5, BG9163, EF6796, RCT123, RCT129, RCT135 and LXS200.

7. The protein-polysaccharide conjugate of claim 6, wherein the N-terminal fragment is comprises a PspA variant of a fragment amino acids 1 to 314 of Rx1314, wherein the methionine at amino acid position 96 is modified to isoleucine.

8. The protein-polysaccharide conjugate of claim 2, wherein the N-terminal fragment is selected from clades 4, 5 or 6 of S. pneumoniae.

9. The protein-polysaccharide conjugate of claim 1, wherein the meningococcal polysaccharide conjugated to the PspA protein is selected from serogroups A, C, Y, W-135 of N. meningitidis.

10. The protein-polysaccharide conjugate of claim 9, wherein the N-terminal fragment further comprises all or part of the proline-rich region of PspA of S. pneumoniae.

11. The protein-polysaccharide conjugate of claim 10, wherein the N-terminal fragment is selected from a PspA of clades 1, 2, 3, 4, 5, or 6 of S. pneumoniae.

12. The protein-polysaccharide conjugate of claim 11, wherein the N-terminal fragment is selected from a PspA of clades 1, 2 or 3 of S. pneumoniae.

13. The protein-polysaccharide conjugate of claim 12, wherein the N-terminal fragment is selected from a PspA of clade 2 of S. pneumoniae.

14. The protein-polysaccharide conjugate of claim 13, wherein the N-terminal fragment is selected from a strain R36A, or variants thereof, including strains Rx1314, Rx1 (ATCC 55834), EF10197, WU2, 0922134, DBL5, BG9163, EF6796, RCT123, RCT129, RCT135 and LXS200.

15. The protein-polysaccharide conjugate of claim 14, wherein the N-terminal fragment is comprises a PspA variant of a fragment amino acids 1 to 314 of Rx1314, wherein the methionine at amino acid position 96 is modified to isoleucine.

16. The protein-polysaccharide conjugate of claim 9, wherein the N-terminal fragment is selected from clades 4, 5 or 6 of S. pneumoniae.

17. An immunological composition comprising one or more protein-polysaccharide conjugates of claim 1.

18. The immunological composition of claim 17, wherein the N-terminal fragment further comprises all or part of the proline-rich region of PspA of S. pneumoniae.

19. The protein-polysaccharide conjugate of claim 17, wherein the N-terminal fragment is selected from a PspA of clades 1, 2, 3, 4, 5, or 6 of S. pneumoniae.

20. The immunological composition of claim 19, wherein the N-terminal fragment is selected from a PspA of clades 1, 2 or 3 of S. pneumoniae.

21. The immunological composition of claim 20, wherein the N-terminal fragment is selected from a PspA of lade 2 of S. pneumoniae.

22. The immunological composition of claim 21, wherein the N-terminal fragment is selected from a strain R36A, or variants thereof, including strains Rx1314, Rx1 (ATCC 55834), EF10197, WU2, 0922134, DBL5, BG9163, EF6796, RCT123, RCT129, RCT135 and LXS200.

23. The immunological composition of claim 22, wherein the N-terminal fragment is comprises a PspA variant of a fragment amino acids 1 to 314 of Rx1314, wherein the methionine at amino acid position 96 is modified to isoleucine.

24. The immunological composition of claim 19, wherein the N-terminal fragment is selected from clades 4, 5 or 6 of S. pneumoniae.

25. The immunological composition of claim 17, wherein the meningococcal polysaccharide conjugated to the PspA protein is selected from serogroups A, C, Y, W-135 of N. meningitidis.

26. The immunological composition of claim 25, wherein the N-terminal fragment further comprises all or part of the proline-rich region of PspA of S. pneumoniae.

27. The immunological composition of claim 26, wherein the N-terminal fragment is selected from a PspA of clades 1, 2, 3, 4, 5, or 6 of S. pneumoniae.

28. The immunological composition of claim 27, wherein the N-terminal fragment is selected from a PspA of clades 1, 2 or 3 of S. pneumoniae.

29. The immunological composition of claim 28, wherein the N-terminal fragment is selected from a PspA of lade 2 of S. pneumoniae.

30. The immunological composition of claim 29, wherein the N-terminal fragment is selected from a strain R36A, or variants thereof, including strains Rx1314, Rx1 (ATCC 55834), EF10197, WU2, 0922134, DBL5, BG9163, EF6796, RCT123, RCT129, RCT135 and LXS200.

31. The immunological composition of claim 30, wherein the N-terminal fragment is comprises a PspA variant of a fragment amino acids 1 to 314 of Rx1314, wherein the methionine at amino acid position 96 is modified to isoleucine.

32. The immunological composition of claim 27, wherein the N-terminal fragment is selected from clades 4, 5 or 6 of S. pneumoniae.

33. The immunological composition of claim 17, wherein the composition comprises two to seven different protein-polysaccharide conjugates of claim 1, where each of the N-terminal fragment is independently selected from a PspA of clades 1, 2, 3, 4, 5, or 6 of S. pneumoniae and each of the meningococcal polysaccharide is independently selected from serogroups A, C, Y, W-135 of N. meningitidis.

34. The immunological composition of claim 33, wherein the composition comprises two to five different protein-polysaccharide conjugates.

35. The immunological composition of claim 34, wherein the composition comprises two to four different protein-polysaccharide conjugates.

36. The immunological composition of claim 35, wherein the composition comprises three or four different protein-polysaccharide conjugates.

37. The immunological composition of claim 36, wherein the composition comprises four different protein-polysaccharide conjugates.

38. The immunological composition of claim 37, wherein each of the four different protein-polysaccharide conjugates comprises the meningococcal polysaccharide from serogroups A, C, Y, W-135 of N. meningitidis.

39. The immunological composition of claim 37, wherein each of the four different protein-polysaccharide conjugates comprises an N-terminal fragment selected from a PspA of clades 1, 2, 3, 4, 5, or 6 of S. pneumoniae.

40. The immunological composition of claim 37, wherein at least one of the four different protein-polysaccharide conjugates comprises an N-terminal fragment selected from a PspA of clades 1, 2, or 3 of S. pneumoniae.

41. The immunological composition of claim 40, wherein at least one of the four different protein-polysaccharide conjugates comprises an N-terminal fragment selected from a PspA of clade 2 of S. pneumoniae.

42. The immunological composition of claim 41, wherein each of the four different protein-polysaccharide conjugates comprises an N-terminal fragment selected from a PspA of clades 1, 2, or 3, of S. pneumoniae.

43. The immunological composition of claim 42, wherein each of the four different protein-polysaccharide conjugates comprises an N-terminal fragment selected from a PspA of lade 2 of S. pneumoniae.

43. The immunological composition of claim 42, wherein each of the four different protein-polysaccharide conjugates comprises an N-terminal fragment selected from a PspA from a strain R36A, or variants thereof, including strains Rx1314, Rx1 (ATCC 55834), EF10197, WU2, 0922134, DBL5, BG9163, EF6796, RCT123, RCT129, RCT135 and LXS200.

44. The immunological composition of claim 43, wherein the N-terminal fragment is comprises a PspA variant of a fragment amino acids 1 to 314 of Rx1314, wherein the methionine at amino acid position 96 is modified to isoleucine.
